SEQUENCE LISTING (5 sequences)

SEQ ID NO: 1 - DNA, 16486 bp, Homo sapiens
  source 1..16486: mol_type = genomic DNA
aaacaaatgcctgatgatccgtcactgtctcccatcatcccgagaggatggtctagttgcaagaaaacaagctcagggctcccactgattctacattatggtgagttgtgtaattatttcattatgtattacaacataataatagaaatgaactgcacactaagtgtaatgtgcttgaatcatcccaaaaccatcctcccgcctccatctgtggaaaaattgtcttccacaaaactggtcccttgtgccaaaaaggttggggactgctatttttttttttctttctgagacagagttttgctcttgttgcccaggctggagtccaatggcgctatcttggctcaccacaacctctgcctcctgggttcaagcgattctccagcctcagcctcccgagtagctgtgattacaggcatgcgccaccacacctggctaattttgtatttttagtagagacagtttctccatgttggtcaggctggtcttgaactcccgatctcaggtgatccgcccgccttggcctcccaaagtgttggggattacaggcgtgagccaccgagcccggccagggactgctgttctaagccacagcgggctgagggcagagctggtgtgagagcgttcaagcttaggcagcaaattaaacagtaaaatggagccgtatctcgaatcaagtctatttttttttccaaaagcaggtatattcctgaaacgtctatttttttttaatctgaaaaacaactttactttaaaaattatctggccaccagaagctcatctgcttcacctctggcaacgccctatagtcctctcacacaaagacaattcttccaaagtgacaaaaatctaacccgcaaactgtttccaagcgaatgcatttttcacaaatgctgaatctaacatttaataaagtgtatatatcatgtatagaagcatttaattaaacatttacttttattttgcagctttccttttgtgttttgtgaattttttaggtctcaaacattttcacagacccacgctgcaggggtgtttttaggggactgggggctcccaattatgtactcgggatgttcccgggggtcctgcctcggtggacccaagcgcacagaccccaggcacgaaggatagaggagctgccccacgccctcaaatactcagccagggctggagaatccttagtcaattcacaaagggggaaactgagtggcaaagggcacccaggaggtcaccgttggaggccccccgggttcaacctaggagctgcaggccgacactggccagggcgcgggcccctcgccgccctcggtcggaaccgccggccgtagctccagggttgcgaccgttaccccacggagctcccgcctccccgcagggaacactcggcacctggtacttctccatgggctctccgggggaccctgggcctcgctccccctgcgtggggcgcctgcgattggaccctggcccaagcatgatcagtagcgaccgcgtacgggcgagcgtccacccctggccccaccccgcgtgggtcagtgggacccgcgggatcctccccgcccgaacggcgccctcgaggcactcaggcgtccctcccgcgcgctggccccagacccccaggtccccgcccccttaaaagggacgcgttccgccgggccgggagggaccgcagggcctgcgcgcggcgggcggggcctccagcgtgagaggcgtggcgtcgagcgcggcgggcgtggcttcgagcggatgggtcgggaggctggggatggcgcgtggcccctttaagacgcaccggggcgggcccgggcggcgcgcggcccctttaaggcttggcctacgtggcagccgctggagccgcagccgacgcggagcgaggccggccgccgggcacttcctgtggaggccgcagcgggtgcgggcgccgacgggcgagagccagcgagcgagcgagcgagccgagccgagcctcccgccgtcgccatgggccagaacgacctgatgggcacggccgaggacttcgccgaccaggtgcggcctgcgggctcctcccccggccccggtcccgacccaggccgcccgggccgctccggagtcggcctcccagcgcagggcgccaggcccgggccgccccctgactccttgttcccacagggggacccccccgcccgggccctctgtcccggctggacagccccaaacccggcctccgggcccagtctcccctgcccgctgggccgctcccggagcccgggcctcggcgccctgcctggctagccgcccccaccctccagccagcccggggcgcgggctgtgggggtttcttcccgggccggcggcgcgagtcctggcgttgggtcgggtctgaggctcgcggggagcgcctcggcgtttggagctgctggggggtccccgcacgtttttcctctcactggggctcccggaggcgggctgggagctcggggacgaacgggggccgggggtcctcccagaggaaagggggcgcgctggcgtgcgccccagctacaggaagccacagcaccttttccttttctctttcccagatgtgctggactcttggggtccctttgaccctgtctgcgtctccctccccgtttggactctgccctgggctggattgagtggggacctgtacacagcctgggaggaggggaagcctatctccctgatagtctccttccagaagtgtccaggtggcattctgagcaacagggaagcctttgaaacaggcggttgggtaactgttgagagcattcgggaactgggtggacaggatccgatcctacaaagtgaagggacacaactcccttccctctttcttgattaagcttgacagaacaaaaaaacaaaaacaaaaggtggtggtggttacgcagatttatgccttttacttctaaaagaatcccgttatgtcgacaaattaacagccttatccccgcagggagagccttgtgtggcccacttgccgttttttcctcggtttacttaatgtgttcgtgtggctttgctagagttttaagtagtgatggttaaatttttaaatttttctggaggtggtagtgagtcatcaaactctgaggcctagacttatcggaaggcttgtatatgtgtggtttaaaaaaaaaagaaaagagtaagtgaccgttttctgatcagctattgaactcttccgttcctcctggggggtgatctctttctagtgtgacctcaccatcggttctcattgcaacactttctgtgagccaatattttaaatttggagggaaaaagtaatcaacttgacgaagcattttttccctaagattctgattttgtgcttaaagacacaattttatttgtttatttatttttatttatttattttttctttgagatggtgttttgaccttgttgcccaggctggagtgcaatggtgtgatctcagcacaccacaacctccaactcctgggttcaagcgattctcctgcctcagcctcccgagtagctgggattacaggcatgcaccgtcacacccagccaattttgtattttttttttttttagtagaaacagggtttctccatgttggtcaggctggtctcgaactcccgacctcaggtggtctgcccacctcagcctcccaaagtgctgggattacaggcgtgagccacggcacccagcctttaaagacacaattttaaaaacaattcatatcaaaagctctcaagaaccaggtagaccttttaggtttgtcagttttcattgacataatttatagtgacccatttatatttctccaagtcgcagctccctaagaagatggctcctcatagctgagggaaggatgagagctacatacactcgctctttgtcctggaaaacctggatttgcaagtgaatttcataaccaaatctagatcaaatgtctccagtcaagctgctgcccactagcaaatgacagactcccctattgacaaaaaaaagttcccagtgtcttcacatactgttttcttcttcttttttttttttttttttttttttttgagacagaatttcgctcaggctggagtgtaatggtgcgatctcggctcactgcaacccccgcctcccggattcaagcaattctcctgcctcagcccgaagtagctgggattacaggcgcctgccaccacgcccagctaatttttttgtatttttagttgagacggggtttccccatgttggccaggctggtttcgaactcccgacctcaggtggtccgcctgccttagcctcccaaagtgctgggattacaggcgtgagccaccctgccctgccttttcttcatgttttgttattatgttttttttggagacagagtcttactttgtcacccaggctggagtgcagtggcgcaatctcatctcactgcaacctccgcctcccgggttcaagctattctcctgcctcagcctcctcagtagctgagatgacaggcccacgccaccacgccccgctaatttttgtgtttttagtagagacagcattttgccatgttgtcatgttgtccaggctggtctcaaacacctgaccttaggtgatccgccttggcctcctaaagttctgggattacatgcctgagccaccgcgcccagccttcatcatgtattctgttatgttttaaatgaaagaagcagtagcttggaactggaaaggctcttgagggggcacagagtcgacctctcccaggttactgaggggaggccaaacctgctcaggctcctagacttgcctgtgaccaagaccaagaaaagccgggtcttggtccccagcgcccccagcccctggtgttgctcttctggcacaagtcaagggaagagcactagcgggtgaaggggctgctcacatacaaaacccaagtgggttaggaccatatctgtaaggcaccatatctgtaaggcagcgcaggctgccgccgtcagatgatctccttaagagtccgtctaggtggtggcagagcatggctgtgggagcttccatgcccttttgtgggcagcttgggagggcagctacttgtactcgcccaaaccccaatcagacagatgcaggcaagtaaacagggttcgaagtcaaaatatggcttcttgtggacagaatctgaccaagcccaggtggggctttgtttctctttattcacatagtacttaaagctgccagaccaggcttttacatgaaaatgacctaatttgaagccgatagaggattaccatggaagtcactgatcagataaagtgacacatccattttttcacatcttcagaaaacaagagcaaaactgaagtgacaagatttgaccagaaagtgtgggctgctgaaagcaagacctaagtaagcttagtttcccaggcccagtgtcttccagtattatgacaatgacattaagtctttcactgggccgggtccatgatagacattccttttgtgaacttcttgactgtaggaggttggccagagcgtcaccatcttcagatgggcagagggaggttggctggccgcactggcttgtggtggcctggagactgagtccgcatcctgttaccccaaaggctgggttcctcactgcttggttgactgatctttccttctggcctgcagaggctggtccacggggacgagcagtctggttagtttctgctggcaggctccccacagttgcatctcttgtctctctgcctctggcatcaagctgcttgctctggcagccagggacagtgtcctcttgctcccaggagccatgctctgtagaaactgctgagacgtgctctatgcaggagcttggcatttggaagccatgggaagggtccagaaactaatgaactcagcaatccaaactaatgtccatgcctgcagctgctcctcattgcctattagacactctgtaaaatgtagcttctaagcagtttatacccataaacagcaatattagcacttaatgggttctgcctggtggctcatcaccaaggtgacagaaaagctgcatggggccttcctctccagtgtgacctttggtgtttttaacctgtcctcaagagtggcagagctagtcctgggccctaggagctggcctcggccctagttgaagtgctggggaagcaaggataggcctgaggacccctgaacttgcaggtggcacaggcactgtgagggtcccgatgaacaccatgctggtgactggtgtggcaaagtcctggtgacagtggcaggctctgtcataccctgccctatatcatgaaggagtggggagagaaggcaaagaggagctgactcacttttctttgtgtgatgatcttaacgggaggttaaaactcactcttcaggggagtgcgggcgaggagcagaggaggttgtggttagcggggctcctcccagaacatggggcccagtcgttctcagttacgtctttcctatagttgggattttgatcacacagttttttagtaaagccaggccatcatctcccacattgggctggcgacatcctttctggccctgcagggtgcagtgtgaacctatagagcagcatgggagcggggctcggggaagttctggagctgcagtgagccagtgagctcgagccagctctgacaggctgatgtgagggaagccgctgggccgcaggtgcttgtttctaggatgtgtgggacctgagttgggctttgaacaaggcagccccagtccttggtgatcaaggaaatctgacctgcctttttcgtcgatgttcacattcaccacgaatttgcaagctgctttcctcaatttctttctgtcctgagtgtgaagagaaagtggagttttctgtgtggcgtgggagctgcaggtggctgtgacagtgatcactggatgcccaggaagtcatctcagggtggatataattaattacgtttctaaaatccccttcagtcgtctaaaataaccacccctttgagagaaatatcagtcttcctgttaagcccctcagatgctgtcactgcttatgaaccatatcctgcttgatggcgcggttctcggagagacttaatgagaagcttggccacagaggtgagctaactgtcatcttcttcctgcagcctgccaggtcaacagccacctctggctctgctgtcgcagttctctaatgggggggagggggactgtcacaagttttatgtgctcgccgtgtttacacaggtcacctgtgtgctggctaccagcaatggccttagtgcctgcagtgcggctgctcttgtctgtcctggggtgctgtgtgtcctgcctgtgttgagggctcttctctgctgtgctgaggggacactggctctgggacgtgttctagagtggcatgacatggttccagtggccaagttgaagtagttgtgaccaacatgcatgtaggaaataaggttaatgttgggccttgtctttaaacgtggctttttcagtgttcagtccaaacagatgctcattaaatggtggccagtttagaggagactgcatcagcctgacttcagccttcagtgtggaccaggcttgcctttgaaaaccaggacagccaagcagtaggtgtgctcagttcagaaggcagtacctagatttttgcatatttttccccaaagccaagagctgtgtctgtttcgtcaccatgtttgcgcagggaaggaggtgtgaattgcctgtcccaccttctctctcttttttgctctttaatcctcttgacccagacactcaaattaagagtcgcagactcattcaaagagacttttaaacactgcctcaatctcgtgggctgtgcacgtagatgagcatgttaaatgcaggtcctgcaactgggggaggcttagaccgcagcccatggctgggctggtttgctcatctcaggccagtgagtggctcacagctctcattttagaagcaagtcagaacacctctggtgtctgtcttgggtatggattgcagtctacctctctgctgctgttggggtccacgccccacacccacaccctgccctgggagtcccaccacggctccgtctcatctcagaggtctggacagcctcctgttgagctcttccttcccagtggttgcttttccccaggttcagatgagataaaggcttgtgtccttgctggggcttcccagctgggccacgatgtgaggccattccttgagagcagtggcacatcgtgtgccacggtggtgtgctttctctcttccatggaatatgggtgattacactgttgctgggcatttagagagcaacggggccatgaggcagaacctggggagggtgtgtggcgtcacctttcccaccacccagtgtgatgcaggttgcgtcacactgtgcacatgggcatcttgtgctggttctctgcattggccctaaaattcacaggggagtatttggggcctttttaaaaataaaaagtgaaacaaggtcttcctcctagtaaagttggaaagcatcttaccaaccattagttcaggtggcctcactctactgcaggatagtagagacactctcaaaaaagtaactgggcgtgtccaggtcacacggtgtgctggctggatggtaccagggcaggatgagatcacagggcctcagaacctccacctgcacttcatctcccaaacccacttggtgattctctgggtatctgtcggtcactgtgatttctcccatcacttccgtggagtcaaaggctgagtaaatgtgtgcagaactgacactgcagggcgcagtgggtttggggagaatggccacctggctttgtaatctctgcctccgttctctgccacatgggagccaatctgcagtgctgacctagtcagggccccacctgacaggctggagggtgacctgaacacccaggggcgtggtaactgagatgggcagcttcagcagtgtctaggactttgaagcaacttttaaaaatgctgaaaaataagaaacacgcatatcttcctttgttccaacagtaggtttagtcagccctaggaggcctgtgcagctgacgttcgctcgcctcccgtgggggtcagcctctcgggtgctctttagactctggcttcccaacatgcgttggtggatgggtcccctgatctttctccgaaggacttgctgtgttacagtatttctttcactttatatctcagttttgcatgggtgataagagaaaagtgcatggggtgaacctgcgtgacatttgtacctggcatccgatagtgcatgttggctacctaacccagccgtcataaatgacctgactcacagcacagcaggaagtcataaatgttttctttccctttgacatttgacagtgatttcacacacttgcgtttcattgctgttgaaacagctgtccatcccatttgttagtgtgttttaatttgctcgagattatgcaggctttgacaaaatcccagcgctttctagccattagcggtaccagcttcatgctgcgctgacggttgctgcacacacagtccccacgtcagatcctgagcttgcaggggagagctccgtcgggctgggaagcgccagccctggtttgcaacagcacacatgcgctgtccagagcctggggcctcctcctgctgctctggagattttcggataactgtaaacgtgatcttactattcagtcaaccatgtggccctctaggtgtggcaccctggctgtcacctaagccaacccttgttctgtcagggtactctgggcgtcactgggcaggctgggaggtccaggcccttggcacctcagccatgtgtgacctgcaggctggggtcaagtcttggcattgcaccatcaggtctgcgaagcctaggggaaccccttcccgctgggtttaatatagccatttgtaaggtgaggggttggaccagagcagcggagatgcctggcaaagtgcagcggtgcctcaggagggaggctgctgctggcatgtcgtgggcagacatgtcgtgcgtcatagcctgcagcacccaggatgacccacggcaaagagtggcctggcctcagatgccagtagtgctgaggtgcagaaacgttggatagatgacctctaagtgccttttcagatgtcattctgtgattccaacttgtaaatgcctttgaccgaaaagagcagctctgtgtcttgtggcagtgccaccttggaagactgtagttgcctgccgaggcactgggggaaatcacggtagcagagtgttagggctgctgccctataggatctgccccagagggcctaacgtcaagtcatgcatctccgggactgatccttggattgttgcatgtgtccagactcacagggtagcgctcaggagcaagcactgagagccccctagtctgccctgggaaggcaccggggctgacaagagctcttgccttccacagcgtctgttcctcacacaccccgcccaaggacgtgcctggcagcggcctggtgctccccgccgcagccacctcacccttgggtttctgtggccctgcagttcctccgtgtcacaaagcagtacctgccccacgtggcgcgcctctgtctgatcagcaccttcctggaggacggcatccgtatgtggttccagtggagcgagcagcgcgactacatcgacaccacctggaactgcggctacctgctggcctcgtccttcgtcttcctcaacttgctgggacagctgagtgagtggctctgcgggtggtcaggattcactgggcgtcttgttccatgatcgttagcagttgggaggctgtgtgtgaactcgggggtctcctggactgctgtcccccctccattcccctcttctccagctcagctgtgccaggccaggagtccggttctgcaagccttggtggggacgcttagtctctagccctgagctcactgctttagagcttgctaaggaaataggacatggtccctgtcagggacccaaagcaggagatgctggagtctggctggcagtccacagtagagcagttaattgcctacctcaggtgggtggctgacttgggagctgattgtgggaaacagcccattctttatgacggcaagggctccactttctggtgtttgtgataagtctagtcagcttaccatgtgtggtccttgcctatcattaattcttccagaagttgtcttttgcttgggtaggtggtttggctgcctctggacctaagggccaggtgaggtagcagtgtatcaagacagtgctccccgccggcacccaccctcaggccccctaacccttgtccttctctccagctggctgcgtcctggtgttgagcaggaacttcgtgcagtacgcctgcttcgggctctttggaatcatagctctgcaggtacacgggccgcgtggtcggggctcccttctctttgctctggtggagcagagcttctgaaaagacatccttgagggttcagtgtcactcccccttcagccccctgcatgtctctgactgcctggagctcctttctcttctggggaagtcgtgagcccaagcagggctgtagaggcaggtcattcggtactgggccatcttttgtcccatttctatgattaccgtgacaagggaagtctcgagcttggctgcagccaggatgctgtcttgttatcacgttgcttcataaagaagaatacttggttattcagaatgaaatgctgggaacagaatatctgaatccacatcagtggtctcagaatcactttgttctctggaacatcatagcacatttcttgagaaatacagaaccacatttttgagcttagatctgtcctcggtctcctacaatggttttgtgtgttttagactccagctgacattggagagacggcctaatgtattatgaagtctgtggaaatgcctttggaaaggctgagactcaagtatctctctcttctttcccctcagacgattgcctacagcattttatgggacttgaagtttttgatgaggtatgtgcattctggttggttttagtatacgctccttctacaggttgagcattgcaaatccaaagtctgaaatgctccaaaatgggaaaaatcttgggcaccaacatgacgctcaaaggaagtgctcattggagcatttcaggttttggattttcagatttgggatgctcagccaataagtatataatgcaaatataccaaaatctgaaacactttgggtcctaagcatttcaggttagggatactcaatctgtatttctaaactcagttaactattggtttgagtagacttctctgggtctaaaccaagcttgtccagcctgtggtccacggggtgaacatggcccaacacaaactcaaaaactttcttaaaacattgagagttttttttgtgatttttaagttttattttttagctcatcacctatcattagcattagtgtattttatgtgtggccaagacagttcttccagggtagcccagggaagccaaaagattggacccccctgtctaaattgtgcctcttagaaacaggttctgaagaggatgttgcagagcccctagaatagtagagactgaagccgtctcttctgacccttatttcacgggtgtggggactccgggtctgagtggaggacagacctgaccacggggacccagctggtcaacactgaagctgtgactaggactcggttgtgtgtgtgttcaggtagcctgtcactgtcgagttgtaaagatacctctgtctcctgagctatgtaaggttttacatgggtgtaactcgagtctgaggtcctgtgcgcatgcctgcctggtcctgcttggcagatcactgggtttggagaaagggaagtaagggccttcttatactggcagcaaggtctccctgcacctggtctcacagacaggccagatcctggtgaggcttagcttccagcccacatatctgttcctcaggaacctggccctgggaggaggcctgttgctgctcctagcagaatcccgttctgaagggaagagcatgtttgcgggcgtccccaccatgcgtgagagctcccccaaacagtacatgcagctcggaggcagggtcttgctggttctgatgttcatgaccctccttcactttgacgccagcttcttttctgtaagtattcctacttgcctgaaccggtctggtttcctgtgaatgctgtccctcccccaccccctccctgctcacccctccctggttgggtattaaccacagcctgggggagctgctgaaatggagcaaacttttttttttttttttttgagacggagtctcgctctgtcccccaggctggagtgcagtgcgtgatctcggctcaccgcaagttccgcctcccgggttcacgccattctcctgcctcagcctcccgagcagctgggactacaggcgcctgccaacacgcctggctaattttttgtatttttagtagagacagggtttcaccgtgttagccaggatggtctcgatctcctgacccctcgtgatctgcctgtctcagcctcccaaattgctgggattacaggcgtgagccaccgtgctcggcggagcaaactttttgatcccattaaaacagaccaaaacatcgtcaatctcttgtgtgtcaagccttctttccagcaaatgaaggccagaacctctgaccttggtggagtgtgcttggcccttggttggccaaggctctccacccgggctccccttactcttctttctttcctcactgttcgtatctttctcaagtaattcctgtgtgtcaggtgtcaggggcaggcctccaggagcttccagagcacacctccggagctgactgagagccctggccgggtagtgtccaggaagagctcctggcgccatggagagaggagagaggatgtttctggcctggatggctgggtgggccaagaagcacttgggaatttagttatggattagagattcatcttggtaaatcacagcctgttactagttgggaacttcagtcttccccaaaccttccacatttcaaagctggtgctttattgcctcctgcctccctttggcttttatctccctacttgcaccagcagccgtctcagcagtagctacatcagcctgatggatagagacttcatgacgtgcagcagctggaacttttgtttataaagcatctcttttcctcacatttattttgaatttcttacgtttcaacctatgcagattgtccagaacatcgtgggcacagctctgatgattttagtggccattggttttaaaaccaagctggctgctttgactcttgttgtgtggctctttgccatcaacgtatatttcaacgccttctggaccattccagtctacaagcccatgcatgacttcctgaaatacgacttcttccagaccatgtcggtgattgggggcttgctcctggtggtggccctgggccctgggggtgtctccatggatgagaagaagaaggagtggtaacagtcacagatccctacctgcctggctaagacccgtggccgtcaaggactggttcggggtggattcaacaaaactgccagcttttatgtatcctcttcccttcccctcccttggtaaaggcacagatgttttgagaactttatttgcagagacacctgagaatcgatggctcagtctgctctggagccacagtctggcgtctgacccttcagtgcaggccagcctggcagctggaagcctcccccacgccgaggctttggagtgaacagcccgcttggctgtggcatctcagtcctatttttgagtttttttgtgggggtacaggagggggccttcaagctgtactgtgagcagacgcattggtattatcattcaaagcagtctccctcttatttgtaagtttacatttttagcggaaactactaaattattttgggtggttcagccaaacctcaaaacagttaatctccctggtttaaaatcacaccagtggctttgatgttgtttctgccccgcattgtattttataggaatagtgaaaacatttagggacacccaaagaatgatgcagtattaaaggggtggtagaagctgctgtttatgataaaagtcatcggtcagaaaatcagcttggattggtgccaagtgttttattgggtaacaccctgggagttttagtagcttgaggcaaggtggaggggcaagaagtccttggggaagctgctggtctgggtgctgctggcctccaagctggcagtgggaagggctagtgagaccacacaggggtagccccagcagcagcaccctgcaagccagcctggccagctgctcagaccagcttgcagagccgcagccgctgtgggcagggggtgtggcaggagctcccagcactggagacccacggactcaacccagttacctcacatggggccttttctgagcaaggtctcgaaagcgcaggccgccctggctgagcagcaccgccctttcccagctgcactcgccctgtggacagccccgacacaccactttcctgaggctgtcgctcactcagattgtccgtttgctatgccgaatgcagccaaaattcctttttacaatttgtgatgccttaccgatttgatcttaatcctgtatttaaagttttctaacactgccttatactgtgtttctctttttgggggagcttaactgcttgttgctccctgtcgtctgcaccatagtaaatgccacaagggtagtcgaacacctctctggcccctagacctatctggggacaggctggctcagcctgtctccagggctgctgcggcccagccccgagcctgcctccctcttggcctctcatccattggctctgcagggcaggggtgaggcaggtttctgctcataagtgcttttggaagtcacctacctttttaacacagccgaactagtcccaacgcgtttgcaaatattcccctggtagcctacttccttacccccgaatattggtaagatcgagcaatggcttcaggacatgggttctcttctcctgtgatcattcaagtgctcactgcatgaagactggcttgtctcagtgtttcaacctcaccagggctgtctcttggtccacacctcgctccctgttagtgccgtatgacagcccccatcaaatgaccttggccaagtcacggtttctctgtggtcaaggttggttggctgattggtggaaagtagggtggaccaaaggaggccacgtgagcagtcagcaccagttctgcaccagcagcgcctccgtcctagtgggtgttcctgtttctcctggccctgggtgggctagggcctgattcgggaagatgcctttgcagggaggggaggataagtgggatctaccaattgattctggcaaaacaatttctaagatttttttgctttatgtgggaaacagatctaaatctcattttatgctgtattttatatcttagttgtgtttgaaaacgttttgatttttggaaacacatcaaaataaataatggcgtttgttgta

SEQ ID NO: 2 - DNA, 22 bp, synthetic construct
  source 1..22: mol_type = other DNA
tgcctacagcattttatgggac

SEQ ID NO: 3 - DNA, 21 bp, synthetic construct
  source 1..21: mol_type = other DNA
tcttcccttcagaacgggatt

SEQ ID NO: 4 - DNA, 21 bp, synthetic construct
  source 1..21: mol_type = other DNA
tgttgccatcaatgacccctt

SEQ ID NO: 5 - DNA, 18 bp, synthetic construct
  source 1..18: mol_type = other DNA
ctccacgacgtactcagc

(19) United States
(12) Patent Application Publication
(10) Pub. No.: US 2026/0117182 A1
(43) Pub. Date: Apr. 30, 2026

(54) SURF4 GENE KNOCKOUT ERYTHROID PROGENITOR CELLS AND METHODS OF ERYTHROID DIFFERENTIATION THEREOF

(71) Applicant: PUSAN NATIONAL UNIVERSITY INDUSTRY-UNIVERSITY COOPERATION FOUNDATION, Busan (KR)

(72) Inventors: Dongjun Lee, Yangsan-si (KR); Jiwon KIM, Yangsan-si (KR); Hee Eun BAE, Busan (KR); Hee Seon CHOI, Yangsan-si (KR)

(73) Assignee: PUSAN NATIONAL UNIVERSITY INDUSTRY-UNIVERSITY COOPERATION FOUNDATION, Busan (KR)

(21) Appl. No.: 19/303,364

(22) Filed: Aug. 19, 2025

Related U.S. Application Data
(63) Continuation of application No. PCT/KR2025/008445, filed on Jun. 18, 2025.

(30) Foreign Application Priority Data
Oct. 24, 2024 (KR) 10-2024-0146578

(51) Int. Cl. C12N 5/078; C12N 5/00; C12N 15/113; C12N 15/86

(52) U.S. Cl. CPC C12N 5/0641; C12N 5/0018; C12N 15/113; C12N 15/86; C12N 2310/14; C12N 2310/531; C12N 2500/25; C12N 2501/125; C12N 2501/14; C12N 2501/39; C12N 2501/999; C12N 2506/11; C12N 2510/00; C12N 2740/15043

(57) ABSTRACT

The present disclosure relates to a SURF4 gene-knockout erythroid progenitor cell and a method of differentiating the same into an erythrocyte, wherein it has been determined that SURF4 gene-knockout cells, in which a SURF4 gene is knocked out in erythroid progenitor cells, express erythroid differentiation markers at a higher rate and undergo erythroid differentiation more rapidly under erythroid differentiation conditions according to the present disclosure, such that the present disclosure provides a method that enables rapid differentiation of erythrocytes using SURF4 gene-knockout erythroid progenitor cells.

### CROSS-REFERENCE TO RELATED APPLICATION

This application is a Continuation of PCT International Patent Application No. PCT/KR2025/008445 filed on Jun. 18, 2025, which claims priority to Korean Patent Application No. 10-2024-0146578 filed on Oct. 24, 2024 which are all hereby incorporated by reference in their entirety.

### SEQUENCE LISTING

This application contains a Sequence Listing submitted via USPTO Patent Center and hereby incorporated by reference in its entirety. The Sequence Listing is named \2280-602.xml, created on Aug. 16, 2025, and 21,658 bytes in size.

### BACKGROUND OF THE INVENTION

The present application is supported by the National Research Foundation of Korea under Ministry of Science and ICT, Ministry of Trade, Industry and Energy, Ministry of Health & Welfare, Ministry of Food and Drug Safety for the following research projects:

1) A study on development of new concept erythrocyte differentiation and proliferation restoration technology through identification of differentiation dynamics of highly efficient mobilized hematopoietic stem progenitor cells based on niche enhancing factors (RS-2023-KH140743).

2) A study on research center for molecular control of cancer cell diversity (2022R1A5A2027161).

3) A study on mechanical study on the differentiation control of immature leukemia/cancer stem cells based on key factors and treatment of blood cancer in the bone marrow microenvironment (RS-2024-00333287).

### 1. FIELD OF THE INVENTION

The present disclosure relates to a SURF4 gene-knockout erythroid progenitor cell and a method for differentiating the same into an erythrocyte.

### 2. DESCRIPTION OF THE RELATED ART

Erythrocytes are the most abundant cellular component of blood, constituting approximately 44% of total blood cells, and primarily function to transport oxygen. Erythrocytes are produced from hematopoietic stem cells through several differentiation stages, where the nucleus is gradually condensed and the cell size decreases after going through burst forming unit erythroid (BFU-E), colony forming unit erythroid (CFU-E), proerythroblast, basophilic erythroblast, polychromatic erythroblast, and orthochromatic erythroblast. After the condensed nucleus is enucleated, it becomes a reticulocyte, and as the remaining RNA and microorganelles are removed and it takes on a biconcave shape, it becomes a mature erythrocyte (RBC). This differentiation process typically takes place over three weeks in vitro, and cell proliferation is limited after the polychromatic erythroblastic stage where mature erythroblasts are predominant.

Erythrocytes are essential in emergency situations such as bleeding and in pathological, medical conditions such as cancer, blood, and surgery. Though erythrocytes are collected through blood transfusions worldwide, there are limits to the transfusion of erythrocytes with suitable conditions, and problems such as contamination with pathogens and viruses occur during the process of collecting and transporting blood, such that various attempts are being developed to provide safe blood. One of the attempts is the creation of artificial blood, the basis of which is the production of erythrocytes in a test tube. A known method for producing erythrocytes in vitro is to use a differentiation medium from regulatory stem cells. However, the current method has a critical shortcoming in terms of large-scale production, with an issue that proper maturation of erythrocytes does not take place.

### SUMMARY OF THE INVENTION

### Problem to be Solved by the Invention

An object of the present disclosure is to provide a method of preparing a SURF4 gene-knockout erythroid progenitor cell and differentiating the same into an erythrocyte.

### Means for Solving the Problem

The present disclosure provides a method for erythroid differentiation comprising: knocking out a Surfeit locus protein 4 (SURF4) gene in erythroid progenitor cells in vitro; and culturing the SURF4 gene-knockout erythroid progenitor cells in an erythroid differentiation medium to differentiate the erythroid progenitor cells into erythrocytes.

In addition, the present disclosure provides an erythrocyte differentiated according to the method.

### Effects of the Invention

According to the present disclosure, it has been determined that SURF4 gene-knockout cells, in which a SURF4 gene is knocked out in erythroid progenitor cells, express erythroid differentiation markers at a higher rate and undergo erythroid differentiation more rapidly under erythroid differentiation conditions according to the present disclosure, such that it is possible to provide a method that enables rapid differentiation into erythrocytes using SURF4 gene-knockout erythroid progenitor cells.

### BRIEF DESCRIPTION OF THE DRAWINGS

FIG. 1 shows an experimental schematic for establishing a SURF4 gene-knockout cell line from erythroid progenitor cells using the Gene silencing system via shRNA.

FIG. 2 shows a result of analyzing whether a SURF4 gene is knocked out in SURF4 gene-knockout cells established according to the present disclosure via a Quantitative RT-PCR.

FIG. 3 shows an experimental schematic for differentiating SURF4 gene-knockout erythroid progenitor cells established according to the present disclosure into erythrocytes.

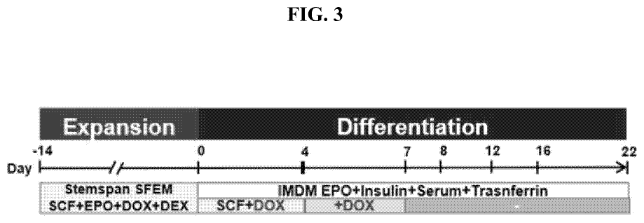

FIG. 4 shows results of analyzing changes in an expression level of erythroid differentiation markers after differentiating SURF4 gene-knockout erythroid progenitor cells established according to the present disclosure into erythrocytes.

### DETAILED DESCRIPTION OF THE INVENTION

The terms used in this specification are selected from general terms that are most widely used currently as much as possible while taking into account the functions of the present disclosure, but these may vary depending on the intention of those skilled in the art, precedents, or the emergence of new technologies. Additionally, in certain cases, there are terms arbitrarily selected by the applicant, and in such cases, meanings thereof will be described in detail in the description of the relevant disclosure. Therefore, the terms used herein should not be simply defined as names of terms, but based on the meaning of the terms and the overall content of the present disclosure.

Unless otherwise defined, all terms used herein, including technical or scientific terms, have the same meaning as commonly understood by those of ordinary skill in the art to which the present disclosure pertains. Terms defined in commonly used dictionaries should be interpreted to have a meaning consistent with those in the context of the relevant art and not be interpreted in an idealized or overly formal sense unless expressly defined in this application.

Hereinafter, the present disclosure will be described in more detail.

The present disclosure provides a method for erythroid differentiation, comprising: knocking out a Surfeit locus protein 4 (SURF4) gene in erythroid progenitor cells in vitro; and culturing the SURF4 gene-knockout erythroid progenitor cells in an erythroid differentiation medium to differentiate the erythroid progenitor cells into erythrocytes.

The progenitor cells are undifferentiated cells with a self-replicability and differentiation capacity, but are ultimately differentiated with a cell type to be differentiated predetermined. As used herein, the term “erythroid progenitor cell,” unless otherwise specified, is intended to include erythroid precursor cells. The erythroid precursor cells refer to all cells involved in the process of erythrocyte formation except mature erythrocytes. Specifically, the erythroid progenitor cell may refer to an erythroid cell before enucleation and may be an immature erythroid progenitor cell or a glycophorin A-positive cell prior to enucleation, where the glycophorin A is a molecule specific to the erythroid lineage. For example, the erythroid progenitor cells may be selected from the group consisting of proerythroblasts, basophilic erythroblasts, polychromatic erythroblasts, and orthochromatic erythroblasts.

The erythroid progenitor cell is cultured in a medium including a stem cell factor (SCF), erythropoietin (EPO), doxycycline (DOX), and dexamethasone (DEX).

The knocking out of the SURF4 gene is to knock out the SURF4 gene (Genebank ID: NC_000009.12) represented by SEQ ID NO: 1 via the Gene silencing system using shRNA, and the SURF4 shRNA is introduced into erythroid progenitor cells by lentivirus.

The SURF4 gene-knockout erythroid progenitor cells are selected from a medium containing puromycin.

In the knocking out of the SURF4 gene, the lentivirus containing an shRNA that complementarily binds to the SURF4 gene represented by SEQ ID NO: 1 is added to the erythroid progenitor cells, followed by culture for 24 to 48 hours.

The differentiating into erythrocytes consists of culturing the SURF4 gene-knockout erythroid progenitor cells in a primary differentiation medium containing erythropoietin (EPO), insulin, transferrin, doxycycline (DOX), and a stem cell factor (SCF) for 4 days; culturing the SURF4 gene-knockout erythroid progenitor cells for 3 days in a secondary differentiation medium containing erythropoietin (EPO), insulin, transferrin, and doxycycline (DOX); and culturing the SURF4 gene-knockout erythroid progenitor cells in a third differentiation medium containing erythropoietin (EPO), insulin, and transferrin.

The differentiating of the erythroid progenitor cells into erythrocytes is completed within 7 to 22 days.

In addition, the present disclosure provides an erythrocyte differentiated according to the method.

Hereinafter, in order to help understand the present disclosure, experimental examples and examples will be described in detail. However, the following experimental examples and examples are only illustrative of the contents of the present disclosure, and the scope of the present disclosure is not limited to the following experimental examples and examples. The experimental examples and examples of the present disclosure are provided to more completely explain the present disclosure to those skilled in the art.

### [Example 1] Establishment of SURF4 Gene-Knockout Cells

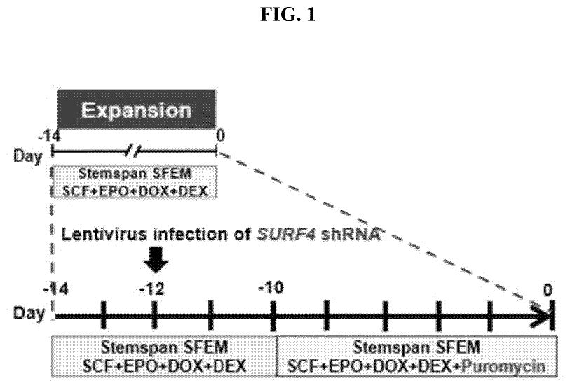

To identify the role of the Surfeit locus protein 4 (SURF4) gene in erythroid differentiation, a SURF4 gene-knockout cell line was established in HUDEP-2 cells, a human erythroid progenitor cell line, using the Gene silencing system via shRNA in the same manner as in FIG. 1. HUDEP-2 cells were cultured in Stemspan SFEM (Stemspan™ Serum-Free Expansion Medium, STEM CELL technologies) medium containing 0.4 μg/ml dexamethasone (DEX, Sigma), 1 μg/ml doxycycline (DOX, Clontech), 50 ng/ml stem cell factor (SCF, R&D systems), and 5 ng/ml erythropoietin (EPO, Peprotech). To knock out the SURF4 gene in HUDEP-2 cells grown in a medium containing cytokines, HUDEP-2 cells were transformed via the spin infection method using SURF4 shRNA lentivirus. After adding 0.8 μg/ml polybrene (Santacruz), a chemical that helps viral infection, to the Stemspan SFEM medium containing cytokines, 1×10⁶ HUDEP-2 cells were suspended in 2 mL of the medium. After dispensing into a 6-well plate, 1 mL of 2×10⁶ TU/ml lentivirus containing SURF4 shRNA (sc-92607-SH, Santa Cruz) was added. Centrifugation was performed for 90 minutes at 20° C. and 2,000 rpm. After 24 hours, the cells were resuspended in 2 mL of fresh cell culture medium containing polybrene, and 1 mL of SURF4 shRNA lentivirus was added, followed by centrifugation once more. After 24 hours, the medium was replaced with Stemspan SFEM medium containing cytokines. After culturing for 24 hours, the medium was replaced with those containing 0.5 μg/ml puromycin and cultured for 5 days to screen only virus-infected cells.

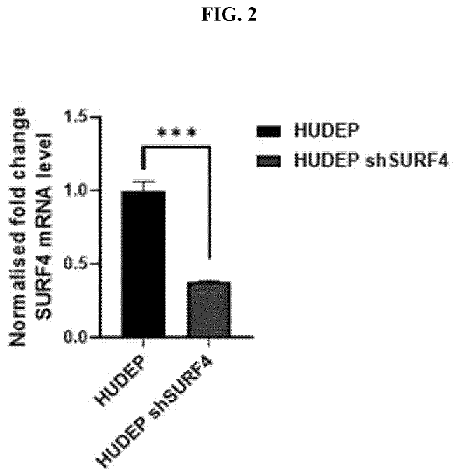

To determine whether the screened HUDEP-2 cells had undergone SURF4 gene manipulation compared to the control group in which no viral transformation took place, the quantitative RT-PCR detection method was used to evaluate whether the SURF4 gene was knocked out. RNA was extracted from cells using the RNeasy plus mini kit (Qiagen). The extracted RNA was reverse-transcribed into cDNA using the SmartGene Compact cDNA Synthesis Kit (SMART GENE). The primers used were the SURF4 forward primer of SEQ ID NO: 2 (TGC CTA CAG CAT TTT ATG GGAC), the SURF4 reverse primer of SEQ ID NO: 3 (TCT TCC CTT CAG AAC GGG ATT), the GAPDH forward primer of SEQ ID NO: 4 (TGT TGC CAT CAA TGA CCC CTT) and the GAPDH reverse primer of SEQ ID NO: 5 (CTC CAC GAC GTA CTC AGC). As shown in FIG. 2, reduction in the expression of SURF4 gene was detected in HUDEP-2 cells applied with the Gene silencing system compared to control cells with no viral transduction via the quantitative RT-PCR.

### [Example 2] Differentiation of SURF4 Gene-Knockout Cells into Erythrocytes

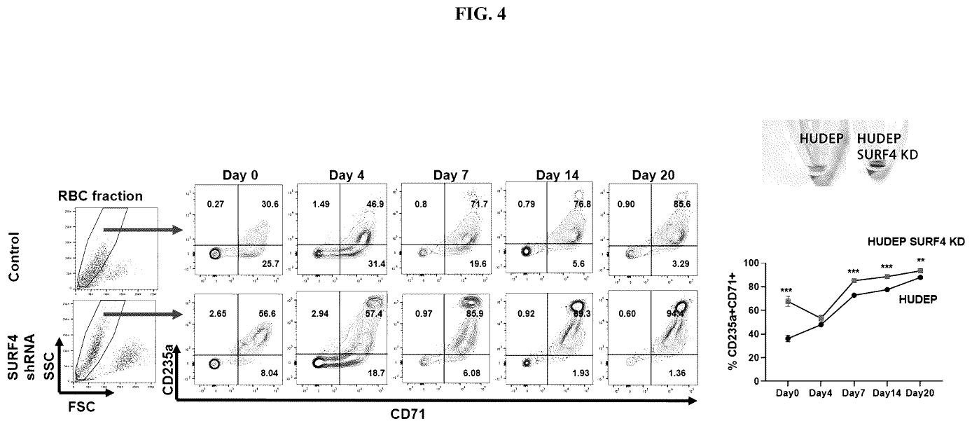

An evaluation was conducted on whether the erythroid differentiation potential of the SURF4 gene-knockout erythroid progenitor cells differs. To differentiate erythroid progenitor cells into erythrocytes, the erythroid progenitor cells were cultured for 4 days in Iscove's Modified Dulbecco's Medium (IMDM) containing 5% human AB serum (Sigma), 400 μg/mL holo-transferrin (Sigma), 10 μg/mL human insulin (Sigma), 5 ng/ml erythropoietin (EPO, Peprotech), 1 μg/mL doxycycline (DOX, Clontech), 41.25 μg/mL heparin (Sigma), and 50 ng/mL stem cell factor (SCF, R&D systems). Afterwards, the cells were cultured for 3 days after removing SCF from the IMDM medium, and then cultured in a medium with DOX removed. Erythroid differentiation potential was monitored at Day 4, Day 8, and Day 12 from the start of differentiation (Day 0). After treatment with fluorescence-labeled antibodies, differences in the expression of erythroid differentiation markers CD235a and CD71 were analyzed via flow cytometry. As shown in FIG. 4, compared to the control group, the expression of erythroid differentiation markers CD235a⁺ and CD71⁺ was found to be at a higher rate, and the erythroid differentiation was carried out more rapidly in SURF4 gene-knockout cells (SURF4 shRNA).

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the attached claims and their equivalents.

The numerical range includes the numerical values defined in the above range. Any maximum numerical limit given throughout this specification includes any lower numerical limit as if the lower numerical limit were explicitly written out. Any minimum numerical limit given throughout this specification includes any higher numerical limit as if that higher numerical limit were explicitly written out. Any numerical limit given throughout this specification will include any better numerical range within the broader numerical range, as if the narrower numerical limit were explicitly written.

What is claimed is:

1. A method for erythroid differentiation, the method comprising:
knocking out a Surfeit locus protein 4 (SURF4) gene in erythroid progenitor cells in vitro; and
culturing the SURF4 gene-knockout erythroid progenitor cells in an erythroid differentiation medium to differentiate the erythroid progenitor cells into erythrocytes.

2. The method of claim 1, wherein the erythroid progenitor cell is cultured in a medium comprising a stem cell factor (SCF), erythropoietin (EPO), doxycycline (DOX), and dexamethasone (DEX).

3. The method of claim 1, wherein the knocking out of the SURF4 gene comprises using the Gene silencing system with shRNA to knock out the SURF4 gene (GenBank ID: NC_000009.12) represented by SEQ ID NO: 1.

4. The method of claim 3, wherein the SURF4 shRNA is introduced into the erythroid progenitor cells by lentivirus.

5. The method of claim 1, wherein the SURF4 gene-knockout erythroid progenitor cells are selected from a medium comprising puromycin.

6. The method of claim 1, wherein the knocking out of the SURF4 gene comprises adding a lentivirus, which comprises an shRNA that complementarily binds to the SURF4 gene represented by SEQ ID NO: 1, to the erythroid progenitor cells, followed by culture for 24 to 48 hours.

7. The method of claim 1, wherein the differentiating into erythrocytes comprises:
(a) culturing the SURF4 gene-knockout erythroid progenitor cells in a primary differentiation medium comprising erythropoietin (EPO), insulin, transferrin, doxycycline (DOX), and a stem cell factor (SCF) for 4 days;
(b) culturing the SURF4 gene-knockout erythroid progenitor cells for 3 days in a secondary differentiation medium comprising erythropoietin (EPO), insulin, transferrin, and doxycycline (DOX); and
(c) culturing the SURF4 gene-knockout erythroid progenitor cells in a third differentiation medium comprising erythropoietin (EPO), insulin, and transferrin.

8. The method of claim 1, wherein the differentiating of the erythroid progenitor cells into erythrocytes is completed within 7 to 22 days.

9. An erythrocyte differentiated by the method according to claim 1.
